(19) United States
(12) Patent Application Publication
(10) Pub. No.: US 2026/0348025 A1
(43) Pub. Date: Nov. 5, 2026

(54) SINGLE-STEP METHOD FOR CONVERSION OF CARBON-DIOXIDE INTO UREA

(71) Applicant: Debayan SAHA, Basking Ridge, NJ (US)

(72) Inventors: Debayan SAHA, Basking Ridge, NJ (US); Shashi RANJAN, Basking Ridge, NJ (US)

(73) Assignee: FARADAY EARTH INC., Wilmington, DE (US)

(21) Appl. No.: 19/486,913

(22) PCT Filed: Jun. 25, 2024
(86) PCT No.: PCT/IB2024/056150

§ 371 Date: Nov. 21, 2025

Related U.S. Application Data
(60) Provisional application No. 63/523,123, filed on Jun. 26, 2023.

(51) Int. Cl. C25B 3/09; C25B 3/26; C25B 11/042; C25B 11/065; C25B 11/081; C25B 13/08; C25B 15/08

(52) U.S. Cl. CPC C25B 3/09; C25B 3/26; C25B 11/042; C25B 11/065; C25B 11/081; C25B 13/08; C25B 15/081; C25B 15/083

(57) ABSTRACT

The embodiments herein provide a single-step method for the conversion of a mixture of gases or flue gas containing CO₂, N₂ and H₂O into Urea using plasma as an energy source. The gas mixture is exposed to high voltage AC or DC or pulsed or subjected to radiofrequency electric field to create plasma state of the said gas mixture in the presence of catalysts. The electrical species (ions, radicals etc.) of gases are introduced in electrically activated or ionized water containing solvated electrons, radicals or ions of Hydrogen and other ions or radicals. The Urea is synthesized in such a reducing water media by co-reduction of Amine ions or ions of Nitrogen Oxides, and ions of oxides Carbon. The invention also discloses a method to utilize electrically activated nano-droplets of water to increase the surface area of interaction between a mixture of gases under a plasma field and water. Additionally, the invention discloses that the single-step method disclosed here can also be utilized for synthesizing Ammonia (NH₃). The disclosed method can help in the reduction of CO₂ emissions from industries or can reduce CO₂ concentration in the air.

### CROSS-REFERENCE TO RELATED APPLICATIONS

This application claims the priority of U.S. Provisional Patent Application No. 63/523,123 with the title “Single-step method for conversion of Carbon-Dioxide into Urea”, and the content of which is incorporated herein by reference in its entirety.

### TECHNICAL FIELD

The present invention relates to the electrochemical production of urea from a mixture of gases such as Carbon dioxide (CO₂), Nitrogen (N₂) and water vapor (H₂O). More specifically, the present invention relates to a single-step conversion of said gases sourced from flue gas or industrial smokestacks into urea thereby reducing the energy consumption and overall cost of urea production.

### BACKGROUND

The problem of global warming is well known. The major factor driving global warming is the rising concentration of CO₂ in the atmosphere, which causes the earth to get warmer due to the greenhouse effect. There are several methods that can capture CO₂ either from the source of production like industries or can be captured directly from air. However, there are challenges in terms of transportation and storage of captured CO₂. Thus, there is a need to develop methods to transform captured CO₂ into useful products.

Currently there have been developments in converting CO₂ into useful products such as chemicals and fuels. These chemicals and fuels when used or burned, the captured CO₂ is emitted back to environment. So, there is a definite need to convert CO₂ into products that can keep it captured for a long time or help in further reduction of CO₂ from the atmosphere. One way of doing that is to convert CO₂ into Urea. Urea is a colorless compound with various agricultural and industrial applications, such as promoting plant growth and further reducing CO₂ from the atmosphere through photosynthesis. Urea also aids in the production of many industrial polymers, such as urea-formaldehyde, urea-melamine-formaldehyde, etc. It may be appreciated that if urea is produced by using flue or exhaust gases, it can reduce carbon footprints by converting carbon dioxide and other gases into a useful product.

The commercially known method utilizes the reaction between ammonia and carbon dioxide at a very high temperature. The well-developed method of synthesizing ammonia involves the reaction of nitrogen and hydrogen gases at a very high temperature and pressure using a catalyst. This process of nitrogen fixation for synthesizing ammonia is known as the Haber-Bosch process. However, this process is accountable for a lot of CO₂ emissions, which does not make the process environmentally friendly.

Another process of synthesizing urea is provided in the US published patent document U.S. Pat. No. 3,406,201A, which discloses the reaction of ammonia with gaseous carbon dioxide under pressure in the range of 100 to 300 atmospheres, at a temperature in the range of 140 °C to 200 °C and at a residence time of 5 to 60 minutes to form a urea melt which contains the urea, water and ammonium carbamate. However, in this method, the carbamate intermediate is formed very rapidly from the reactants under elevated pressures and the decomposition of the ammonium carbamate to urea and water takes place. Since the carbamate is not completely converted to water and urea, it is necessary to decompose the carbamate out of the urea-water mixture. Moreover, there are some limitations in toxicity and corrosiveness of the reaction mixture, and the adverse effect of water on the rate of urea formation.

The commercially known methods for the synthesis of urea using carbon dioxide and nitrogen gases require a first conversion of nitrogen to ammonia, followed by a conversion of ammonia to urea using carbon dioxide gas, thereby requiring multiple steps for synthesizing urea from carbon dioxide. Hence, there is a need for synthesis of urea in a one-step process from easily available gases such as CO₂, N₂ and water vapor, especially from the flue and industrial exhaust gases. Also, there is a need for a system for synthesizing urea from such mixture of gases which consumes less energy.

### OBJECTIVES OF THE INVENTION

The primary objective of the present invention is to provide a single-step method for electrical or electrochemical production of urea from a mixture of easily and inexpensively available gases such as carbon dioxide (CO₂), nitrogen (N₂) and water vapor (H₂O).

Another objective of the present invention is to provide a system for synthesizing urea from flue gases, which reduces greenhouse gases.

Yet another objective of the present invention is to synthesize urea from a mixture of gases including carbon dioxide, nitrogen, and water vapor obtained from different sources.

Yet another objective of the present invention is to synthesize urea from a mixture of gases including carbon dioxide, nitrogen, and water vapor obtained from exhaust industrial gases.

Yet another objective of the present invention is to provide a cost-effective method of converting carbon dioxide into urea.

Yet another objective of the present invention is to provide a method of synthesis of urea by reacting flue gases or gaseous mixture (containing carbon dioxide, nitrogen, and water vapor) with nano-droplets of water.

Yet another objective of the present invention is to provide a system for synthesizing urea from flue gas which consumes less energy and is inexpensive.

These and other objects and advantages of the embodiments herein will become readily apparent from the following detailed description taken in conjunction with the accompanying drawings.

### SUMMARY OF THE INVENTION

The various embodiments herein provide a single-step method for electrical or electrochemical production of urea from a mixture of easily and inexpensively available gases such as Carbon dioxide (CO₂), Nitrogen (N₂) and water vapor (H₂O). The embodiments herein also provide a system for synthesizing Urea directly from flue gas or gas containing CO₂, N₂ and H₂O in a single-step process which consumes less energy. The embodiments herein also provide a system for synthesizing Urea from flue gases, which reduces greenhouse gases.

According to the embodiments disclosed herein, the present invention relates to the field of carbon capture and utilization. The present invention discloses a single-step cost-effective method of converting flue gas into Urea leveraging carbon-di-oxide (CO₂) Nitrogen (N₂), and Water Vapor (H₂O) present in the flue gas or effluent gas coming out of the industries. The system disclosed in the present invention is a purely electrical method and can be applied at normal room temperature and atmospheric pressure without any need for energy expensive high temperature and pressure. In one disclosed embodiment non-thermal, non-equilibrium cold plasma is utilized to provide energy to create ions and radicals of N₂, CO₂ and H₂O. These radicals and ions of nitrogen and hydrogen (hydrogen radicals derived from H₂O vapor) are allowed to react over a catalyst surface to promote the formation of N—H bonds which are subsequently pushed in an electrically activated water medium where ions and radicals of CO₂ react with N—H based intermediaries to form Urea.

According to another embodiment herein, a system for converting a mixture of gases or flue gas or industrial effluent gas into Urea in a single-step process using plasma as an energy source consists of a gas-mixing system; a flow regulator; a plasma catalytic reactor; an activated water reaction chamber; a gas separation unit; and a urea filtration system. The gases N₂, CO₂, H₂O/H₂ or flue gas are mixed in a gas-mixing system. The appropriately mixed gases are provided, in a controlled manner using a flow regulator to the plasma catalytic reactor subjected to a High Voltage (HV) electric field to create a plasma of said gases. The plasma-treated gases are bubbled into the activated water reaction chamber to dissolve them into water. The undissolved gases are sent to a gas separation unit to recover any valuable compound, especially ammonia (NH₃). The rest of the gases are recirculated in the gas-mixing system. The urea is synthesized in the activated water reaction chamber. The synthesized urea produced in water in the said chamber is filtered out from the water using a Urea filtration system and the water is recycled back to the activated water reaction chamber.

According to another embodiment herein, the plasma catalytic reactor chamber consists of multiple sections or at least three sections: a first section; a second section; and a third section. The first section comprises a plasma generator which induces the excitation of gas molecules or breaking bonds of molecules to produce radicals and ions. Different types of plasma systems including, but not limited to, a Dielectric Barrier Discharge (DBD) plasma or a Gliding Arc plasma, obtained by HV AC or DC energy source are used in the first section. The second section comprises of a lower energy density plasma obtained by similar plasma generating systems as mentioned in the first section, or different plasma generating systems such as Radio frequency plasma and/or Microwave plasma obtained by HV AC or DC energy source. The second section also comprises a catalyst. The catalyst drives the reaction between Nitrogen and Hydrogen molecules. The catalyst is selected from a group consisting of metal-based, metal oxide-based nanomaterials, and Metal-Organic framework (MOF). The third section comprises multiple pins connected with a HV DC supply to create a pin-jet plasma over the water surface to inject high energy electrons into the water. Such highly energized electrons electrically activate water by creating a plethora of solvated electrons in water. The third section may comprise a catalyst to promote N—H and C—N bond formation. A part of the third section is submerged in water and comprises of an air bubbler to create nano/micro sized gas bubbles in the water. The air bubbler consists of a plurality of nano/micro sized holes. The bubbler may also contain multiple pins and is connected to an HV DC power source to create a pin-jet plasma over the activated water surface at the site where it is injecting gas bubbles in the activated water. The plasma generation is governed through the variation of energy source, energy supply, voltage, current and frequency. Different sources of power for generating plasma can be selected from a group consisting of High Voltage DC, High Voltage High-Frequency AC, Pulsed DC or AC.

According to another embodiment herein, the amount of urea produced depends on the usage of CO₂ used. The final weight of the urea obtained is in a range of 0.1-1.3 times the weight of the CO₂. In general, a molar stoichiometric ratio of CO₂:N₂:H₂O, 1:1:2, might be optimal for Urea production. Additionally, the invention disclosed herein may be utilized for the production of Ammonia (NH₃) in the absence of CO₂ or when the amount of CO₂ is very low.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The present invention will be better understood after reading the following detailed description of the presently preferred aspects thereof with reference to the appended drawings, in which the features, other aspects and advantages of certain exemplary embodiments of the invention will be more apparent from the accompanying drawings in which:

FIG. 1 is a diagram illustrating a system for the synthesis of urea from CO₂, N₂, and H₂O in a single-step process using plasma as a source of energy, according to one embodiment herein.

FIG. 2 is a diagram illustrating a Plasma Catalytic Reactor along with an Activated Water Reaction Chamber of the system shown in FIG. 1.

FIG. 3 is a diagram illustrating another embodiment of an Activated Water Reaction Chamber with an integrated Electrolysis setup.

FIG. 4 is a diagram disclosing another embodiment of the single-step method of converting a mixture of gases or flue gas containing CO₂, N₂, and H₂O vapor using nano-droplets of water as an electrically activated water reaction media.

FIG. 5 is a diagram illustrating a system for the synthesis of urea from CO₂, N₂, and H₂O using nano-droplets of water as an electrically activated water reaction media.

Although the specific features of the embodiments are shown in some drawings and not in others. This is done for convenience only as each feature may be combined with any or all of the other features in accordance with the embodiments herein.

### DETAILED DESCRIPTION OF THE INVENTION

The following description describes various features and functions of the disclosed system and methods with reference to the accompanying figures. In the figures, similar symbols identify similar components, unless context dictates otherwise. The illustrative aspects described herein are not meant to be limiting. It may be readily understood that certain aspects of the disclosed system, method and apparatus can be arranged and combined in a wide variety of different configurations, all of which may not necessarily have been described herein, but should be considered part of the invention disclosed hereinafter.

These and other features and advantages of the present invention may be incorporated into certain embodiments of the invention and will become more fully apparent from the following description or may be learned by the practice of the invention as set forth hereinafter. Accordingly, those of ordinary skill in the art will recognize that various changes and modifications of the embodiments described herein can be made without departing from the scope of the invention. In addition, descriptions of well-known functions and constructions are omitted for clarity and conciseness.

The terms and words used in the following description are not limited to the bibliographical meanings, but, are merely used to enable a clear and consistent understanding of the invention. Accordingly, it should be apparent to those skilled in the art that the following description of exemplary embodiments of the present invention are provided for illustration purpose only and not for the purpose of limiting the invention.

It is to be understood that the singular forms “a”, “an” and “the” include plural referents unless the context clearly dictates otherwise.

It should be emphasized that the term “comprises/comprising” when used in this specification is taken to specify the presence of stated features, integers, steps or components but does not preclude the presence or addition of one or more other features, integers, steps, components or groups thereof.

In the following detailed description, reference is made to the accompanying drawings that form a part hereof, and in which the specific embodiments that may be practiced is shown by way of illustration. These embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The term “High Voltage” (HV) herein refers to the voltage of the AC or DC power source used in this invention to apply an electric field in the range of 0.5 KV to 50 KV.

The present invention relates to systems and methods for electrochemical production of urea from a mixture of gases. The various embodiments of the present invention herein provide methods and systems for the electrochemical production of urea from a mixture of gases such as Carbon dioxide (CO₂), Nitrogen (N₂) and water vapor (H₂O), which are easily available. Such a mixture of gases is commonly present in flue gas or exhaust gas coming out of smokestacks from industries. In some embodiments, the mixture of gases may be obtained from other sources also. This reduces energy consumption and the overall cost of urea production, thereby reducing the emission of greenhouse gases.

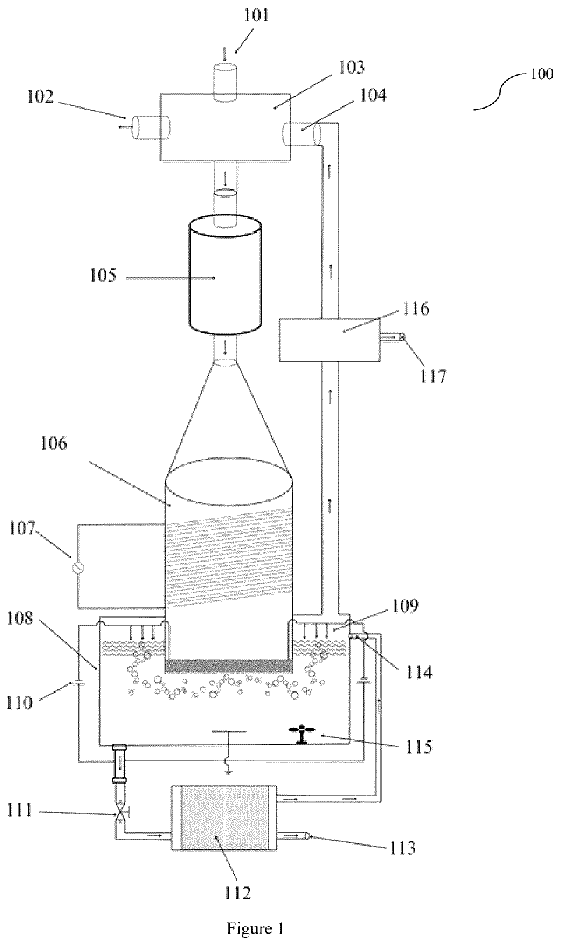

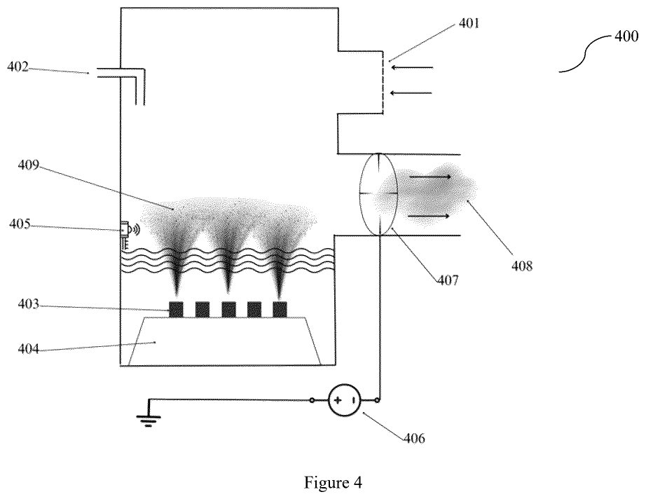

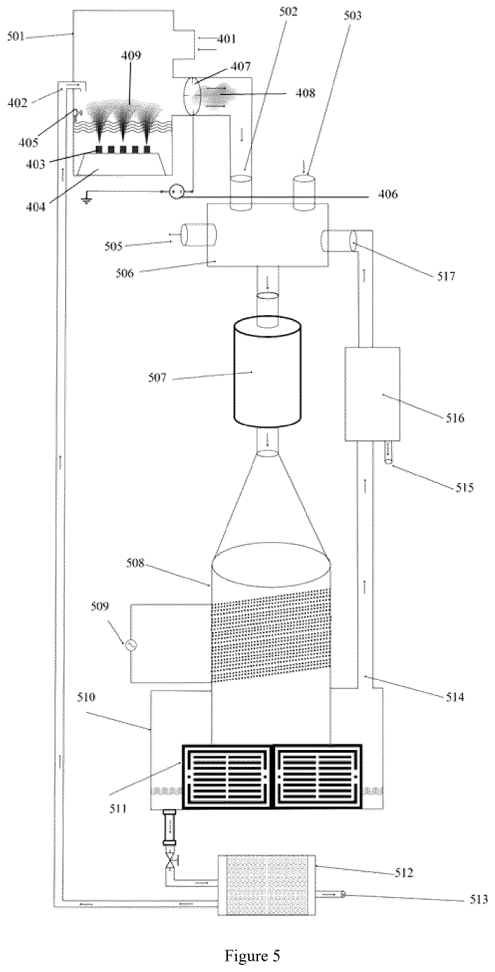

According to some embodiments disclosed herein, systems and methods for a single-step conversion of flue gas or a mixture of gases containing carbon dioxide (CO₂), Nitrogen (N₂) and Water vapor (H₂O) into Urea are disclosed. The methods disclosed in these embodiments herein are purely electrical and can be applied at normal room temperature and atmospheric pressure without any need for energy expensive high temperature and pressure. The embodiments utilizes non-thermal, non-equilibrium cold plasma to provide energy to create ions and radicals of N₂, CO₂ and H₂O. These ions can react over a catalyst surface and then in an electrically activated water medium. The electrically activated water preferably acts as a reducing catalyst. One of the ways of achieving the electrical activation of water is to bombard water with high energy electrons by using gas plasma that generates solvated electrons and other electrically charged species such as radicals and ions in water as shown in FIGS. 1-3. On the other hand, the activation of water may be performed by generating nano-sized water droplets with increased surface area, which may be utilized as a reducing agent to react with plasma-treated gases to form urea as shown in FIGS. 4-5.

The gas mixture comprising at least three components, CO₂, N₂, and H₂O (or other sources of H₂ such as methane or directly using H₂ gas) or each of the separate gas stream or any combination thereof to essentially provide Carbon, Oxygen, Nitrogen and Hydrogen for formation of Urea molecule. The said gas mixture or flue gas is exposed to high voltage AC or DC or pulsed or subjected to radiofrequency electric field to create a plasma state of the said gas mixture. In the plasma state, ions, radicals or other electrically excited and unstable or intermediary species are formed from the said constituents of the gas mixture. The said electrical species (ions, radicals etc.) of N₂ and H₂ (hydrogen radicals derived from H₂O vapor) are allowed to react over a catalyst surface to promote the formation of N—H bonds which are subsequently pushed in an electrically activated water medium containing solvated electrons, radicals or ions of Hydrogen, where ions and radicals of CO₂ react with N—H based intermediaries to form Urea.

Accordingly, in the first embodiment, the present invention discloses a method for synthesis of urea comprising of:
i) introducing a mixture of gases comprising CO₂, N₂, and H₂O into a gas mixing module (103) to obtain a gas mixture;
ii) passing the gas mixture through a mass flow controller (105) into a plasma catalytic reactor (106), wherein the plasma catalytic reactor (106) is powered by a High Voltage AC or DC power source (107) to produce the plasma-treated gases comprising ions and radicals of CO₂, N₂ and H₂O;
iii) passing said plasma-treated gases obtained in step (ii) through a catalyst in a plasma catalytic reactor (106) to promote the formation of amines (N—H bonds);
iv) generating a plurality of bubbles (213) of the plasma-treated gases using an air bubbler (211);
v) supplying a high voltage power supply (110) to a plurality of sharp pins (109) configured to create a pin-jet plasma to inject high energy electrons in the water, to obtain activated water having higher electrochemical reduction potential as compared to the reduction potential of standard hydrogen electrode, which is considered as zero;
vi) injecting the plurality of bubbles (213) of the plasma-treated gases obtained from step (iv) into a water reaction chamber (108), wherein a part of said reactor (106) is submerged in the activated water of the water reaction chamber (108) to dissolve a portion of the bubbled gases (213) in the activated water of the water reaction chamber to produce urea and a plurality of gases including ammonia (NH₃) in the water reaction chamber (108) and to circulate the undissolved gases to a gas-separation module (116);
vii) separating the urea obtained in step (vi) by passing the water containing urea through a urea filtration module (112) and recycling the water back through a channel (114) to the said chamber (108); and
viii) separating the ammonia (NH₃) produced in the water reaction chamber (108) through a channel (117) and recycling unused bubbled gases to the gas mixing module (103) through a channel (104).

The disclosed method is implemented by a system (100) according to one embodiment of the invention. FIG. 1 illustrates a system for the synthesis of urea from a mixture of gases or a flue gas containing CO₂, N₂, and H₂O in a single step using plasma as a source of energy wherein the flue gas, N₂ and H₂O is introduced through an inlet channel or pipe (101) in a gas-mixing module (103). Any excess gases are purged out through an outlet (102). The CO₂ can be obtained from industrial effluent gases while N₂ can be obtained from air and H₂ can be derived from water. The Gases are mixed by a gas-mixing module (103) to generate a desired concentration of each component in the gas mixture that can be transformed into a plasma state, wherein the concentration of each component gas is measured through sensors such as sensors used for measuring CO₂, N₂ and humidity. The gas-mixture is supplied into a Plasma Catalytic Reactor (106) through a mass flow controller (105) that controls the mass flow rate of the gas mixture. The Plasma Catalytic Reactor (106) is powered by a High Voltage AC or DC power source (107). The mixture of gases flowing through the plasma catalytic reactor (106) is subjected to the high voltage electric field to produce a plasma that is further bubbled to an activated water reaction chamber (108), wherein a part of the said reactor (106) is submerged in the activated water of the said chamber (108). The plasma catalytic reactor (106) also contains a plurality of sharp pins (109) to create a pin-jet plasma over the surface of the activated water and is powered by a high voltage DC power supply (110). The urea is synthesized in the activated water reaction chamber (108) and is transferred into a Urea filtration module (112) through a pump (111). The urea is separated out of the water and collected through an outlet (113) and the water is recycled back through a connecting pipe (114) to the chamber (108). In an embodiment, a stirrer (115) may be provided to mix or stir reactants in the chamber (108). The dissolved part of the bubbled plasma-treated gases in the activated water reaction chamber (108) reacts to form urea and is collected in the urea filtration module (112) while the undissolved gases are channeled to a gas separation module (116). The valuable components, especially Ammonia (117) are separated out while other gases are recycled through a pipe (104) to the gas-mixing module (103).

An embodiment of the present invention is to provide one pot synthesis of urea from gas mixtures containing carbon dioxide, nitrogen and water vapor, in which urea is synthesized without any intermediate separation. The terms “single-step synthesis”, “one pot synthesis” and “single pot synthesis” can be interchangeably used throughout the present invention. In the one pot synthesis method, multiple reaction intermediates are generated successively, and the reaction occurs in a single step, thereby avoiding lengthy separation processes.

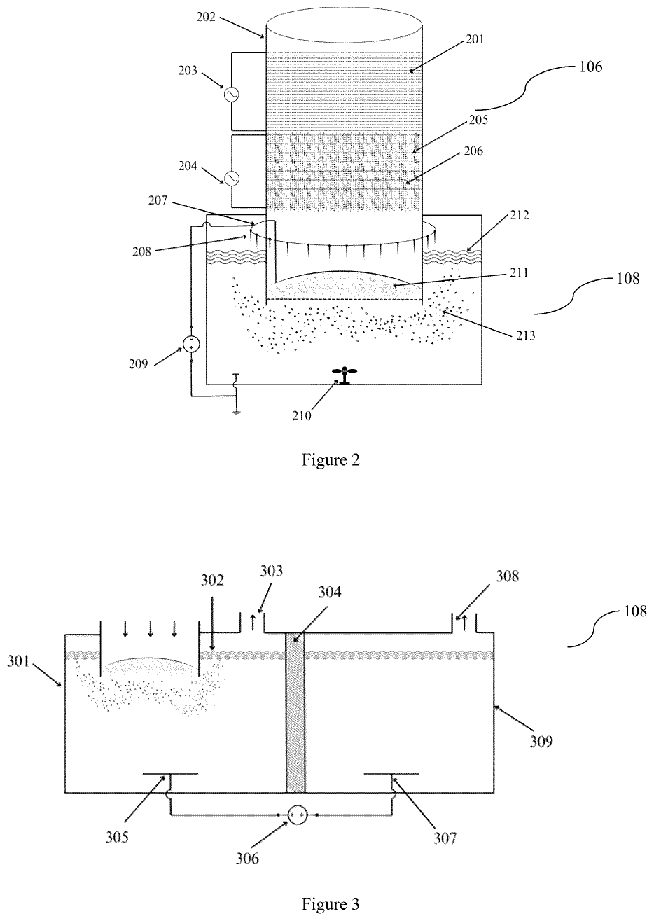

FIG. 2 illustrates an embodiment of the Plasma Catalytic Reactor (106) and the Activated Water Reaction Chamber (108) as shown in FIG. 1. In an embodiment, the plasma catalytic reactor (106) consists of multiple sections or at least three sections: a first section (201); a second section (205); and a third section (207).

The first section (201) comprises a plasma generator (202) connected to a High Voltage AC or DC supply (203), which is configured to generate a high energy density plasma by exciting the gas molecules or breaking the bonds of gas molecules to produce intermediary species in form of ions and radicals. In an exemplary embodiment, the intermediary species i.e., ions and radicals generated by the excitation of the gas mixtures or obtained from recycled gas mixtures may comprise one or more ions and/or radicals including, but not limited to CO, CO⁻, CO₂*, CO*, CO⁺, CO₂ ⁺, O*, NH₂, NH, NH₃ ⁺, NH₂ ⁺, NH₄ ⁺, NH⁺, NH₃*, NH₂*, NH*, OH⁻, OH*, H₂O*, H*, H⁻, H⁺, N₂ ⁺, N₂*, where * indicates a radical. In another exemplary embodiment, the plasma generator (202) used in the first section (201) of the Plasma Catalytic Reactor (106) including, but not limited to, Dielectric Barrier Discharge (DBD) plasma, Gliding Arc plasma obtained by the high voltage (HV) AC or DC.

The second section (205) is a plasma generating system comprises a catalyst (206) and is connected to an High Voltage AC or DC supply (204), which is configured to generate a lower energy density plasma as compared to the energy density of plasma generated in the plasma generator in the previous section (201). The catalyst (206) is configured to drive reactions between Nitrogen and Hydrogen molecules. In an exemplary embodiment, the catalysts may be selected from a group consisting of metal-based, metal oxide-based, nanomaterials, and Metal-Organic framework (MOF). The plasma generating system in section (205) is connected with a high voltage (HV) AC or DC energy source (204) and is configured to generate a low energy density plasma. In an exemplary embodiment, the plasma generating system in section (205) may be similar to the plasma generator in section (201) or may be a different plasma generating system such as Radiofrequency plasma and/or Microwave plasma obtained by HV AC or DC energy source (204).

The third section (207) comprises a plurality of pins (208) connected with a High Voltage (HV) DC supply (209) to create pin-jet plasma over the water surface (212) to inject highly energized electrons in the water stored in the water reaction chamber (108). Such highly energized electrons electrically activate the water (212) by creating an excess amount of solvated electrons in the water. In an embodiment, the third section (207) may comprise a catalyst to promote N—H and C—N bond formation. A part of the third section (207) is submerged in water (212) and comprises an air bubbler (211) to create nano and/or micro sized gas bubbles (213) in the water (212). The air bubbler (211) consists of a plurality of nano and/or micro sized holes formed at the bottom to create nano and/or micro sized gas bubbles, wherein the diameter of the nano and/or micro sized holes may be in the range of 1 nanometer to 100 micrometres.

In an embodiment, the present invention discloses a method of synthesis of urea, wherein the method of generation of plasma by the plasma catalytic reactor (106) comprises of:
producing a high energy density plasma by a first section (201) comprising:
   i) generating a high energy density plasma using a plasma generator (202) connected to a high voltage (HV) AC or DC supply (203);
   ii) exciting the gas molecules of CO₂, N₂, and H₂O to produce intermediary species including ions and radicals thereof;
producing a low energy density plasma by a second section (205) comprising:
   iii) generating a low energy density plasma using a plasma generation system connected to a high voltage (HV) AC or DC energy source (204) and containing at least one catalyst (206);
   iv) performing a plurality of reactions between Nitrogen and Hydrogen ions produced in step (iii) to form amines such as ammonia (NH₃) by the at least one catalyst (206);
   v) supplying a high voltage DC power source (209) through a plurality of pins to create a pin-jet plasma of high energy electrons over the water surface;
injecting high energy electrons and generating bubbles (213) into the water reaction chamber (108) by a third section (207), a part thereof is submerged in water (212) comprising:
   vi) electrically activating water by creating the excess amount of solvated electrons in water to obtain activated water;
   vii) creating pluralities of nano/micro sized gas bubbles (213) in the water reaction chamber (108) using the air-bubbler (211); and
   viii) stirring the mixture of bubbles in the activated water in the water reaction chamber (108) using a stirrer (210) for mixing the gaseous compounds in water (212) to obtain urea.

In another embodiment, the air bubbler (212) may also contain pins and is connected to the HV DC power source (209) to create pin-jet plasma over the water surface at the site where it is injecting gas bubbles (213) in the water. In some embodiments, a stirrer (210) may be implemented in the water reaction chamber (108) for mixing gaseous compounds in the water (212). The plasma generation is governed through the variation of energy source, energy supply, voltage, current and frequency.

Another embodiment of the Activated Water Reaction Chamber (108) as illustrated in FIG. 3, comprises of a first compartment (301), a second compartment (309), at least two electrodes for low voltage electrolysis including a cathode (305) and an anode (307), and an ion exchange membrane (304). The chamber (108) is separated by the membrane (304) by dividing said chamber (108) into two compartments, i.e., the first compartment (301) and the second compartment (309). The chamber (108) comprises of water (302), wherein a part of the plasma catalytic reactor (106) is submerged in the first compartment (309) and the membrane (304) allows selective movement of the ions especially Hydrogen ions from the second compartment (309) to the first compartment (301). The hydrogen ions facilitate the formation of Urea. The ion exchange membrane (304) may be selected from a cation exchange membrane, an anion exchange membrane, an ion selection membrane, or a polyelectrolyte membrane. In an exemplary embodiment, the ion exchange membrane (304) may be selected from, but not limited to, Perfluorosulfonic acid (PFSA) membranes, Polybenzimidazole (PBI) membranes, Polystyrene-divinylbenzene copolymers, Sulfonated polyether ether ketone, and polyelectrolyte membranes. The cathode may be selected from, but not limited to, Platinum (Pt)/Palladium (Pd) based catalyst on carbon black. The anode may be selected from a group comprising, but not limited to Ruthenium dioxide (RuO₂) or Iridium dioxide (IrO₂).

The urea is synthesized in the reaction chamber (108) in water (302) and is extracted by pumping water (308) out into the urea filtration module (112), whereas the unreacted bubbled gas (303) which may contain Ammonia (NH₃) is sent for gas separation (116) for recovery of the NH₃.

In an embodiment, a method for synthesis of urea by electrolysis of water in a water reaction chamber (108) is disclosed, comprising of:
i) performing electrolysis of water in the water reaction chamber (108) containing a cathode (305), an anode (307), and an ion exchange membrane (304), wherein the cathode (305) is arranged in a first compartment (301) separated from the anode (307) arranged in a second compartment (309) by an ion exchange membrane (304);
ii) allowing the movement of Hydrogen ions from the second compartment (309) to the first compartment (301) to initiate the formation of urea; and
iii) producing urea in the water reaction chamber (108) in water (302) and extracting urea into the urea filtration module (112).

According to another embodiment herein, a direct single-step synthesis of Urea may be achieved in micro and/or nano-droplets of water. FIG. 4 shows an embodiment of a water chamber, wherein water is converted into electrically activated nano-droplets and each droplet acts as an activated water reaction chamber (108). The method of converting water into nano-droplets includes the process of providing air by a fan (401) and water by a pump (402) arranged in the device module (400). An ultrasonic wave generator (403) is arranged on a support (404) configured to shear bulk water into micro droplets (409) while maintaining the level of water by a level sensor (405). The size of micro droplets of water may range from 0.5 to 50 micrometers. The micro droplets of water (409) are subjected to an HV electric field (407) controlled by a power source (406) to generate nano-droplets of water (408), wherein the size of nano-droplets of water may range from 1 to 500 nanometers. The nano-droplets of water are allowed to react with the plasma-treated gases with highly increased surface area in the plasma catalytic reactor (106). The conversion of water into nano-droplets allows increase in the surface area of the activated water droplets for increased interaction of gases and water, which allows for better diffusion of the plasma generated species including ions and radicals from gases to the activated water droplets. The Urea produced in nano-droplets is precipitated along with the precipitation of the nano-droplets of the water. The ultrasonic wave generator (403) generates physical shearing which generates ions and radicals of hydrogen. The physical shearing methods for generating ions and radicals in water may be selected from a group consisting of ultrasonic waves, high-pressure shearing, and water splashing (Lenard effect).

In another embodiment of the present invention, a system and a method for synthesizing urea from a mixture of gases including carbon dioxide, nitrogen, and hydrogen may be provided as shown in FIG. 5, wherein a direct single-step synthesis of Urea is achieved using micro or nano-droplets of water obtained in accordance with the embodiment of FIG. 4. The system and method for producing urea as illustrated in FIG. 5, wherein water is converted into electrically activated nano-droplets. For instance, the module (400) of FIG. 4 or a chamber (501) of FIG. 5 is provided to produce nano-droplets of activated water, which is allowed to enter a gas mixing module (506) through an inlet (502) where flue gas containing CO₂ and a mixture of N₂ and H₂ are provided through an inlet (503). Any excess gas is purged out through an outlet (505). The gas mixing module (506) is configured to mix said gases to form a composition required for the formation of urea, wherein the composition of the mixture is determined using sensors for measuring amounts of CO₂, N₂ and humidity. A mass flow controller (507) is configured to control the mass flow rate of the gas mixture composition which is further provided to a Plasma Catalytic Reactor (508). Said gas mixture is exposed to a high voltage AC or DC or pulsed or subjected to radiofrequency electric field to create a plasma state of the said gas mixture. In the plasma state, ions, radicals or other electrically excited and unstable species are formed from the said constituents of the gas mixture. The reactor (508) is powered by a High Voltage AC or DC power source (509), wherein the nano-droplets of water are allowed to react with plasma-treated gases with highly increased surface area in the plasma catalytic reactor (508). The Urea produced in nano-droplets is precipitated using a water droplet precipitator (511) and the precipitated water is collected in the water bath (510), wherein the water droplet precipitator may be selected from the group of condensers, water traps etc. The rest of the gases are bubbled in the precipitated water for further dissolution of plasma-treated gases in the water. The urea produced is filtered out in the urea filtration module (512) and is collected from the outlet (513). The undissolved gases are directed to the gas-separation module (516) through a pipe (514), and the separated gases such as ammonia are collected from the outlet (515), while the rest of the gases are channeled through (517) to the gas mixing module (506).

In an alternative embodiment of the present invention according to FIG. 5, the present invention discloses a method for synthesis of urea using electrically activated micro/nano-droplets of water, comprising of:
i) providing air by a fan (401) and water by a pump (402) in a chamber (501);
ii) generating micro water droplets (409) using an ultrasonic wave generator (403);
iii) passing the micro water droplets generated in step (ii) through a high voltage electric field (407), to generate nano-droplets of water (408);
iv) adding CO₂ and N₂ to nano-droplets of water (408) in a gas mixing module (506) to obtain a gas mixture;
v) passing the gas mixture obtained in step (iv) through a mass flow controller (507) into a plasma catalytic reactor (508), wherein the plasma of the gas mixture is obtained comprising ions and radicals, which react over a catalyst to drive reaction for production of urea within the nano-droplets of water;
vi) producing urea in the form of nano-droplets, wherein nano-droplets of water is precipitated into bulk water using a water droplet precipitator (511) and the precipitated water is collected in the water bath (510); and
vii) filtering the urea obtained from step (vi) in the urea filtration module (512).

According to one embodiment, the present invention provides a system for converting a mixture of gases or flue gas into urea in a single-step process using plasma as an energy source, consists of a gas-mixing module (103) to connect the flue gas containing pipes, inlet (101) and outlet (102); a mass flow regulator or controller (105) to control the flow of gases; a plasma catalytic reactor (106); an activated water reaction chamber (108); a plurality of sharp pins (109) mounted on the plasma catalytic reactor (106) to create pin-jet plasma over the surface of the water and to inject electrons to the water; a gas separation unit (113); a pump (111); a Urea filtration module (112); and a second gas separation module (116) to collect the undissolved gases.

According to one embodiment herein, the weight percentage of CO₂ ranges 1-40% w/w. Water vapour is in a range of 2-80% w/w. The amount of urea produced depends on the usage of CO₂ used. The final weight of the Urea obtained is in a range of 0.1-1.3 times the weight of the CO₂.

According to one embodiment herein, the following reactions occur. Step 1 excites N₂, CO₂, and H₂O bonds using proprietary plasma technology. Step 2 forms radicals like N, NH, NH₂ and CO. Step 3 yields the Urea, after the following reactions:
2NH₃⁻+CO₂→NH₂COONH₄+2e⁻
2NH₃+CO₂^{-→NH}₂COONH₄+2e⁻
2NH₃⁻+CO→CO(NH₂)₂(s)+H₂+2e⁻
2NH₂⁻+CO→CO(NH₂)₂(s)+2e⁻

The embodiments also disclose the process of plasma catalysis to convert a gas mixture comprising Nitrogen, Carbon-dioxide, Hydrogen or water vapor into Urea or Ammonia (NH3).

According to one embodiment herein, different methods of generating non-thermal electric plasma can be used which include but are not limited to, Dielectric Barrier Discharge (DBD) plasma, Microwave plasma, Radio Frequency Plasma, Gliding Arc plasma, Corona Discharge plasma within the same plasma catalytic reactor as disclosed in FIG. 2. The selection of plasma type within the said reactor is designed to selectively excite or break molecular bonds to direct the catalytic reaction in the desired direction. It also discloses the use of thermal plasma.

According to one embodiment herein, plasma helps in breaking a chemical bond, ionizing a molecule and exciting the molecule electronically or vibrationally. Such effects allow the molecules or electrical specials to react with the catalyst. Different types of plasma have different degrees of such effect. It needs to be optimized as per the system design and selection of catalyst.

According to one embodiment herein, the plasma catalytic reactor is designed to have variable energy density and plasma properties such as different filaments or afterglow energy, shape or spacing. These variations are dynamically controlled as per the requirement. The energy supply to different regions of the plasma catalytic reactor (106), can be continuous or intermittent or with variable values which can be controlled through an algorithm or a software. For example, the energy density in the initial part of the tube is higher which can allow molecular bonds of gases to be broken. In the later part, the energy density is lower to allow the catalytic reaction to happen.

According to one embodiment herein, two plasma catalytic reactors are used in parallel. In one of the said reactors, a mixture of N₂ and H₂O vapor/H₂ is subjected to the plasma catalytic reactor and is bubbled through the electrically activated water in the activated water chamber to form ammonium ion (NH₄+) or Nitrates or Nitrites in the water. In another plasma catalytic reactor, a separated CO₂ gas stream is passed and is bubbled through the water to catalyze the reaction of ions or radicals of CO₂ with NH₄⁺ or NH₃ to form Urea. The system is not limited to the sequential introduction of plasma-treated gas mixture in water, any combination and simultaneous mixing of all required gases can also be undertaken. Appropriate catalysts such as metal catalysts, organic base catalysts or biological catalysts are used to catalyze the reaction. The disclosed method can help in the reduction of CO₂ emissions from industries or can reduce CO₂ concentration in the air. The CO₂ in the process can be obtained from industrial effluent gases while N₂ can be obtained from air and H₂ can be derived from water. The embodiments disclose a method of controlled mixing of appropriate gases or gas mixtures such as flue gas or effluent gas from industry with air or N₂ and water vapor or H₂. The goal is to achieve the desired concentration of each component in the gas mixture that can be transformed into a plasma state. The embodiments disclose that H₂ can be obtained from water vapor in air, liquid water or any other source of H₂ such as methane N₂ can be obtained from air by separating it from Oxygen, but it doesn't limit the source of N₂.

According to one embodiment herein, the plasma catalytic reactor design is such that the energy density is higher towards the annular region. Thus, air containing N₂ and H₂O is introduced towards the annular part while CO₂-rich gas is introduced in the core. Both of these gas streams flow in the laminar flow. N₂ is broken by the higher energy density while CO₂ is broken by the lower energy. In this way, overall energy is minimized.

According to one embodiment herein, different types of plasma effects like excitation, ionization, and dissociation within the same plasma type such as DBD plasma are achieved using different combinations of micro-discharge and after flows which can also be controlled through variation of energy supply, voltage, current and frequency. Electrodes with special plasma generating dots can be designed to achieve such phenomena.

According to one embodiment herein, different sources of power can be used for generating plasma which may include but is not limited to, High Voltage DC, High Voltage High-Frequency AC, Pulsed DC or AC.

According to one embodiment herein, a system to minimize the recombination of ions or radicals to form the original gas molecule is disclosed. For example, by plasma treatment, Carbon-dioxide (CO₂) converts into Carbon monoxide (CO) and Oxygen radicals are formed. CO and Oxygen radicals can recombine to form CO₂. In the disclosed method, the CO₂ gas molecule is adsorbed onto a solid adsorbent material. The plasma-treated gas is directly bombarded on the adsorbed gas to initiate a reaction of radicals to the gas molecule for a further breakdown of the gas molecule. For example, Oxygen radicals from the above example can react with adsorbed CO₂ to generate further CO. The disclosed method increases the efficiency of the conversion of gas molecules into suitable forms for further reaction to form useful compounds. In yet another way, the plasma-treated gases are quickly reacted with water or other material (membrane, chemicals etc.) to scavenge Oxygen and hydroxyl radicals to reduce the chances of the said recombination reaction.

According to one embodiment herein, other methods are disclosed to minimize the recombination of ions or radicals to form the original gas molecule as mentioned earlier. It includes but is not limited to, designing purposeful morphology to enhance the chances of reaction of Oxygen radicals with vibrationally excited CO₂ at the periphery of the plasma flame. In another way, introducing Carbon Dioxide not treated through plasma directly into the plasma-treated gas mixture containing CO and O such that the O reacts with this freshly introduced CO₂ to form CO and hence results in lesser recombination back to CO₂ and more CO₂ conversion by reacting with the CO₂ not treated with plasma. In yet another way, introducing the plasma-treated gas quickly into the water to cool it down to diminish the recombination reaction of ions and radicals.

According to one embodiment herein, the embodiments also disclose uniquely designed beads or nanoparticles for achieving three (improving the plasma) important functionalities; first, increasing the local concentration of a particular gas, second, increasing the total surface or enhancing the quality of plasma to increase the reaction efficiency/speed, and third, catalyzing reaction of plasma treated gases for the synthesis of the required compound. For example, CO₂ from air is captured on specially designed beads and is concentrated on beads. The beads are made up of dielectric material and help in the generation of Dielectric Barrier Discharge (DBD) plasma for enhanced reaction of other gases in the presence of plasma and it catalyzes the reaction of plasma-treated gases CO₂ and N₂ and H₂. Such an arrangement can be integrated into the water system to form Urea in the present disclosed embodiments.

According to one embodiment herein, the purpose of these beads is to increase the surface area where the catalyst can be applied. The increased surface area allows more gases to come in contact with the catalyst which can help in higher conversion of flue gases to urea. These beads can be made of glass or plastic such as polystyrene etc. The size of beads may vary from a few microns to a few millimeters.

According to one embodiment herein, beads coated with catalyst are dynamically moved within the plasma catalytic reactor (106) and their movements are controlled by mechanical or magnetic field to allow full utilization of catalytic surface and allow better or controlled flow of gases inside the reactor. It also helps in changing the plasma characteristics dynamically to reduce energy consumption and increase the yield.

According to one embodiment herein, the embodiments disclose a system for increasing the residence time of gases within a plasma catalytic reactor by creating a vortex of gases inside the chamber. Such vortices can be produced by, but are not limited to, designing a gas outlet with a smaller diameter than the reactor diameter, a controlled valve system that changes its orifice size as required etc.

According to one embodiment herein, water in the Activated Water Reaction Chamber (108), can be saturated with solvated electrons by forcing high-energy electrons into water. This can be achieved by but is not limited to, directing a stream of electrons from an electrode maintained at high voltage negative potential. Electrons can be directed in the water by keeping the high voltage counter positive potential inside the water where the thickness of the water column above the positive electrode is minimal and water is timely dispersed away to carry the solvated electrons to the bulk water.

According to one embodiment herein, a thin film of water is generated on the walls of the plasma catalytic reactor (106), to capture electrons generated in the plasma reactor that do not react with any gas molecule. The energy of such electrons is captured in water in the form of solvated electrons.

According to one embodiment herein, two types of plasma, negative field, and positive field, are used where some of the gases are passed through negative field plasma while others are passed through positive field plasma or vice-versa before they are allowed to react in water.

According to one embodiment herein, the catalyst can be used at different reaction pathways to control the reaction dynamics and the final product. The synergy of plasma and the catalytic reaction is controlled to produce intermediates of reaction and direct them to produce the final product. This is done to reduce overall energy requirements. For example, the embodiments disclose a controlled amount of water vapor in the reactor to produce radicals of NH, and NH₂ but inhibit the formation of NH₃, so that energy is not spent in breaking NH₃ again. Overall, the application of a catalyst at the surface of DBD plasma for enhanced conversion of N₂ into its ions and radicals, while another type of catalyst is used inside water for conversion of reactants into Urea.

According to one embodiment herein, a magnetic field is utilized to enhance the effects of the said plasma or to manipulate the effective field of effects of the said plasma. It is also used for the separation of ions or radicals based on the electric charge on these radicals as the magnetic field affects the movement of electrically charged particles.

According to one embodiment herein, the embodiments disclose a method to control the reaction between different gases by manipulating the parameters of the plasma system such as energy density, time of exposure, field intensity, electron density, etc. Such control can be achieved using computer-based algorithmic control through a software.

According to one embodiment herein, the algorithmic control can be automated through the use of artificial intelligence and machine learning methods, wherein the control system can automatically study different parameters including electric parameters as well as parameters related to gases such as the composition of the gas mixture.

According to one embodiment herein, the unreacted activated gases escaping out of the water (109) are recycled to utilize the activated molecules and save overall energy. The activation energy is reduced in subsequent passes.

According to one embodiment herein, the embodiments disclose a system of automated maintenance of catalyst over the bead surface. It is achieved by maintaining the catalyst coating on the beads by plasma treatment of beads and then spraying the catalyst powder automatically within the reactor based on the dynamic movements of beads. This helps in the easy maintenance of the reactor.

The main objective of the embodiments of the present invention is to produce Urea directly from the Flue gas coming out of industries which burn hydrocarbon-based fuel (Natural gas, Coal, Oil etc.). The flue gas comprises CO₂, N₂ and H₂O vapor. Water vapor (H₂O) acts as the source of H₂. The embodiments herein are not limited to synthesize urea from Flue gas and can generate urea if gases (N₂, CO₂ and H₂) are introduced individually. Moreover, the core point is to convert the mixture of gases in Urea in a single-step process.

The present invention helps in reducing CO₂ emissions from industries or can reduce CO₂ concentration in the air. The present invention provides a system for a single-step cost-effective method for the production of urea from flue gas or a mixture of gases containing carbon-di-oxide (CO₂), Nitrogen (N₂) and Water vapor (H₂O).

The method of the present invention is purely electrical and can be applied at normal room temperature and atmospheric pressure without any need for energy expensive high temperature and high pressure.

The present invention provides a carbon capturing method for conversion of Carbon dioxide (CO₂) into urea that reduces energy consumption and also reduces greenhouse gas emissions.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments.

It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims. Although the embodiments herein are described with various specific embodiments, it will be obvious for a person skilled in the art to practice the embodiments herein with modifications.

We claim:

1. A method for synthesis of urea comprising of:
i) introducing a mixture of gases comprising CO₂, N₂, and H₂O into a gas mixing module (103) to obtain a gas mixture;
ii) passing the gas mixture through a mass flow controller (105) into a plasma catalytic reactor (106), wherein the plasma catalytic reactor (106) is powered by a High Voltage AC or DC power source (107) to produce the plasma-treated gases comprising ions and radicals of CO₂, N₂ and H₂O;
iii) passing said plasma-treated gases obtained in step (ii) through a catalyst in a plasma catalytic reactor (106) to promote the formation of amines (N—H bonds);
iv) generating a plurality of bubbles (213) of the plasma-treated gases using an air bubbler (211);
v) supplying a high voltage power supply (110) to a plurality of sharp pins (109) configured to create a pin-jet plasma to inject high energy electrons in the water to obtain activated water having higher electrochemical reduction potential as compared to the reduction potential of standard hydrogen electrode, which is considered as zero;
vi) injecting the plurality of bubbles (213) of the plasma-treated gases obtained from step (iv) into a water reaction chamber (108), wherein a part of said reactor (106) is submerged in the activated water of the water reaction chamber (108) to dissolve a portion of the bubbled gases (213) in the activated water of the water reaction chamber to produce urea and a plurality of gases including ammonia (NH₃) in the water reaction chamber (108) and to circulate the undissolved gases to a gas-separation module (116);
vii) separating the urea obtained in step (vi) by passing the water containing urea through a urea filtration module (112) and recycling the water back through a channel (114) to the said chamber (108); and
viii) separating the ammonia (NH₃) produced in the water reaction chamber (108) through a channel (117) and recycling unused bubbled gases to the gas mixing module (103) through a channel (104).

2. The method as claimed in claim 1, wherein the gas is a mixture of CO₂, N₂, and H₂O or a flue gas comprising CO₂, N₂, and H₂O, or a mixture thereof.

3. The method as claimed in claim 1, wherein the generation of plasma by the plasma catalytic reactor (106) comprises:
producing a high energy density plasma by a first section (201) comprising of:
i) generating a high energy density plasma using a plasma generator (202) connected to a high voltage (HV) AC or DC supply (203);
ii) exciting the gas molecules of CO₂, N₂, and H₂O to produce intermediary species including ions and radicals thereof;
producing a low energy density plasma by a second section (205) comprising of:
iii) generating a low energy density plasma using a plasma generation system connected to a high voltage (HV) AC or DC energy source (204) and containing at least one catalyst (206);
iv) performing a plurality of reactions between Nitrogen and Hydrogen ions produced in step (iii) to form amines such as ammonia (NH₃) by the at least one catalyst (206);
v) supplying a high voltage DC power source (209) through a plurality of pins to create a pin-jet plasma of high energy electrons over the water surface;
injecting high energy electrons and generating bubbles (213) into the water reaction chamber (108) by a third section (207), a part thereof is submerged in water (212) comprising of:
vi) electrically activating water by creating the excess amount of solvated electrons in water to obtain activated water;
vii) creating pluralities of nano/micro sized gas bubbles (213) in the water reaction chamber (108) using the air-bubbler (211); and
viii) stirring the mixture of bubbles in the activated water in the water reaction chamber (108) using a stirrer (210) for mixing the gaseous compounds in water (212) to obtain urea.

4. The method as claimed in claim 3, wherein the plasma generator (202) of the first section (201) is selected from a group of Dielectric Barrier Discharge (DBD) plasma, Gliding Arc plasma and the plasma generating system of the second section (205) is selected from a group of the plasma generators of the first section (201), Radio frequency plasma, Microwave plasma.

5. The method as claimed in claim 3, wherein at least one catalyst (206) is selected from a group consisting of metal-based, metal oxide-based, nanomaterials, and Metal-Organic framework (MOF).

6. The method as claimed in claim 3, wherein the intermediary species of ions and radicals comprising from the group selected from CO, CO⁻, CO₂*, CO*, CO⁺, CO₂ ⁺, O*, NH₂, NH, NH₃ ⁺, NH₂ ⁺, NH₄ ⁺, NH⁺, NH₃*, NH₂*, NH*, OH⁻, OH*, H₂O*, H*, H⁻, H⁺, N₂ ⁺, N₂*.

7. The method as claimed in claim 3, wherein the urea obtained is in a range of 0.1-1.3 times the weight of CO₂ used in producing urea.

8. A method for synthesis of urea by electrolysis of water in a water reaction chamber (108) comprising of:
i) performing electrolysis of water in the water reaction chamber (108) containing a cathode (305), an anode (307), and an ion exchange membrane (304), wherein the cathode (305) is arranged in a first compartment (301) separated from the anode (307) arranged in a second compartment (309) by an ion exchange membrane (304);
ii) allowing the movement of Hydrogen ions from the second compartment (309) to the first compartment (301) to initiate the formation of urea; and
iii) producing urea in the water reaction chamber (108) in water (302) and extracting urea into the urea filtration module (112).

9. The method as claimed in claim 8, wherein the ion exchange membrane (304) is selected from a group consisting of Perfluorosulfonic acid (PFSA) membranes, Polybenzimidazole (PBI) membranes, Polystyrene-divinylbenzene copolymers, Sulfonated polyether ether ketones and polyelectrolyte membrane.

10. The method as claimed in claim 9, wherein the cathode is selected from, Pt/Pd based catalyst on carbon black.

11. The method as claimed in claim 9, wherein the anode is selected from Ruthenium dioxide (RuO2), Iridium dioxide (IrO2).

12. A method for synthesis of urea using electrically activated micro/nano-droplets of water, comprising of:
i) providing air by a fan (401) and water by a pump (402) in a chamber (501);
ii) generating micro water droplets (409) using an ultrasonic wave generator (403);
iii) passing the micro water droplets generated in step (ii) through a high voltage electric field (407), to generate nano-droplets of water (408);
iv) adding CO₂ and N₂ to nano-droplets of water (408) in a gas mixing module (506) to obtain a gas mixture;
v) passing the gas mixture obtained in step (iv) through a mass flow controller (507) into a plasma catalytic reactor (508), wherein the plasma of the gas mixture is obtained comprising ions and radicals, which react over a catalyst to drive reaction for production of urea within the nano-droplets of water;
vi) producing urea in the form of nano-droplets, wherein nano-droplets of water is precipitated into bulk water using a water droplet precipitator (511) and the precipitated water is collected in the water bath (510); ands
vii) filtering the urea obtained from step (vi) in the urea filtration module (512).

13. A system for the synthesis of urea, comprising:
a gas mixing module (103) containing inlet pipes (101) to introduce gas mixtures containing CO₂, N₂, H₂O and/or flue gas;
a mass flow controller (105) connected to the gas mixing module (103) to control the mass flow of the gas mixture;
a plasma catalytic reactor (106) comprising at least one catalyst and connected to the mass flow controller (105), wherein the plasma catalytic reactor (106) is powered by a High Voltage (HV) AC or DC power source (107) to generate a plasma of ions and radicals;
an activated water reaction chamber (108) in which a part of the plasma catalytic reactor (106) is submerged;
a plurality of sharp pins (109) mounted on the plasma catalytic reactor (106) to create pin-jet plasma of high energy electrons over the surface of water;
a pump (111) connecting the activated water reaction chamber (108) and a urea filtration module (112);
the urea filtration module is configured with an outlet pipe for urea (113) and a connecting pipe (114); and
a gas separation module (116) to separate ammonia from the undissolved gases.

14. The system as claimed in claim 13, wherein the water reaction chamber (108) comprises:
a. a first compartment (301) configured with a cathode (305),
b. a second compartment (309) configured with an anode (307), and
c. and an ion exchange membrane (304) separating the first compartment (301) and the second compartment (309),
wherein the ion exchange membrane (304) allows movement of Hydrogen ions from the second compartment (309) to the first compartment (301).

15. A system for the synthesis of urea, comprising:
i) a fan (401) and a pump (402) installed in a chamber (501) for introducing air and water in the chamber (501);
ii) an ultrasonic wave generator (403) for generating micro water droplets (409);
iii) a high voltage electric field (407) power source to generate nano water droplets from micro water droplets (409);
iv) a gas mixing module (506) to obtain a gas mixture of CO₂, N_{2,} and activated water nanoparticles;
v) a mass flow controller (507) to control flow of the gas mixture;
vi) a plasma catalytic reactor (508), wherein plasma of the gas mixture is obtained comprising ions and radicals, which react over a catalyst to drive reaction for production of urea;
vii) a water droplet precipitator (511) that precipitates the nano-droplets of water containing urea produced within such nano-droplets;
viii) a water chamber (510) for collection of water obtained from the precipitation of water from nano-droplets;
ix) a urea filtration module (512) to filter out urea obtained; and
x) a gas separation unit (516) for separating ammonia from the unreacted gases.
